(19) United States
(12) Patent Application Publication
(10) Pub. No.: US 2024/0074921 A1
(43) Pub. Date: Mar. 7, 2024

(54) HYDROPHOBIC COMPONENTS FOR DISPOSABLE ABSORBENT ARTICLES

(71) Applicant: Baby's Breath Cotton Diapers Inc., Madison, NC (US)

(72) Inventor: Nicole Marie Richards, Madison, NC (US)

(73) Assignee: Baby's Breath Cotton Diapers Inc., Madison, NC (US)

(21) Appl. No.: 18/189,968

(22) Filed: Mar. 24, 2023

Related U.S. Application Data
(60) Provisional application No. 63/374,576, filed on Sep. 5, 2022.

(51) Int. Cl. A61F 13/49; A61F 13/15

(52) U.S. Cl. CPC A61F 13/49; A61F 13/15804; A61F 2013/49092

(57) ABSTRACT

A disposable absorbent article, such as a diaper, having non-woven hydrophobic leg cuffs and an inner layer comprising natural fibers derived from the flower/fruit/seed/stalk of a plant that is harvested seasonally and a treatment applied to at least one surface thereof that imparts hydrophobicity. The treatment includes at least one compound selected from the group consisting of waxes, urethanes, silicones, fluorocarbons, and non-fluorochemical repellants.

### CROSS REFERENCE TO RELATED APPLICATIONS

This application claims priority to U.S. Patent Provisional Patent Application No. 63/374,576, filed on Sep. 5, 2022; the entire content of the provisional application is incorporated by reference herein.

### FIELD OF THE INVENTION

The present invention relates to a hydrophobic top sheet/inner layer of fluid-absorbing structures (absorbent articles), such as diapers, incontinence products, feminine hygiene products, etc., and more particularly, relates to disposable absorbent articles with a hydrophobic top sheet/inner layer of fluid-absorbing structures, such as diapers, incontinence products and feminine hygiene products, etc., which are promoted as more sustainable using natural fibers.

### BACKGROUND OF THE INVENTION

Articles used to absorb waste body fluids such as urine, blood, menses, and the like, are used in large quantities worldwide. These articles are commonly referred to as being “absorbent articles”. Typically, these articles are found in the form of diapers, and other like products, such as, feminine hygiene products and incontinence products, which have become increasingly popular as a modern convenience and necessity.

Absorbent articles in the form of diapers generally come in two varieties, namely, reusable cloth diapers and disposable diapers. Modern cloth diapers come with liners, including flushable liners, which hold the waste and permit disposal into a toilet or the like. Disposable diapers greatly increased in popularity following the introduction of superabsorbent polymers (SAP) such as TRO C, sodium polyacrylate, polyacrylamide copolymer (without limitation) in diapers in the mid-80s and today, an estimated 90% of U.S. parents use disposable diapers.

A basic disposable diaper includes the following components: (a) an inner layer or top sheet that sits next to the baby's skin and thus, serves as the initial layer that contacts waste fluids and/or solids; this inner layer or top sheet is most often made of some kind of petroleum-based plastic or plastic-treated material; however, some green diaper companies are using plant-based plastic (aka bioplastic) or hydrophilic cotton blends to provide this layer; (b) an absorbent core that absorbs and holds the fluids. Most absorbent cores are made of “fluff,” where fluff is made from wood pulp fibers, corn- or wheat-based materials and can include crystals and/or conventional superabsorbent materials (SAP) that are dispersed throughout the fluff such that the fluff serves to distribute the fluid, while the SAP absorbs and “locks” the fluid in the core away from the baby; (c) a waterproof outer shell or in some cases semi-outer shell layer (e.g., a film) that provides a waterproof coating/layer. This coating or layer is most often made of some kind of petroleum-based plastic or plastic-treated material; however, some green diaper companies are using plant-based plastic (aka bioplastic) to provide this layer; and (d) a film covering layer (back sheet) that provides a different feel than the waterproof coating/layer. Such film covering layers are most often made of some kind of petroleum-based plastic or plastic-treated material like the film layer; however, some green diaper companies are using plant-based plastics (aka bioplastics) and/or cellulosic nonwoven fabrics to provide this layer. This back sheet layer is typically only used to offer a softer feel for the external surface of the absorbent article than one would achieve with only the film layer exposed as the outermost layer.

The top sheet is most commonly prepared from woven, nonwoven, or porous formed-film polyethylene or polypropylene materials and is extremely hydrophilic. As such, fluid quickly absorbs through the top sheet and into the core where it is locked away in the fluff (SAP). These common plastics do not have a rewet issue typically as they are not affected by moisture regain being synthetic materials. Back sheet materials typically comprise flexible polyethylene sheets along with the film barrier. The back sheet can be formed of the same material as the top sheet; however, in some cases, the back sheet further includes the film barrier (that acts as a fluid barrier for leak proofing the diaper) on the inner surface of the back sheet, either as one component or where the film is bonded to the inner layer of the back sheet. The outer surface of the back sheet comprises the back sheet material (e.g., nonwoven polyethylene), which is soft to the touch and thus the film barrier is concealed in a traditional diaper construction.

Due to the widespread use of disposable diapers, large quantities are used each year and disposal of these large quantities is an ever-enlarging environmental problem. Most of the commercially available disposable diapers consist largely of plastics based on polypropylene and polyethylene which are not sustainably sourced. Manufacturers of same typically have no or little consideration for the environmental impact their production of plastics has on the planet. On a more positive note, some green plastic manufacturers are starting to incorporate recycling and or bio-polymers that have additional treatments such as enzymes embedded into the plastic to help the bacteria begin to degrade the polymer matrix after discard. Regardless of which is used, recycling or bio-polymers with enzymes, these green processes still produce a negative or harmful environmental impact by way of pollutants emitted in the extrusion process and/or in the manufacturing processes.

In fact, synthetic polymers are a significant challenge because they are often disposed of in landfills where they will remain for centuries into the future slowly leaking toxins into soil as time passes. Most notably, the average disposable diaper can take 500 years to decompose and contains petroleum, plastics, perfumes, wood pulp, and dioxins.

In particular, in many if not most disposable diapers, the liquid permeable surface material and the leak-proof backing material are not sustainable (i.e., fulfilling the needs of current generations without compromising the needs of future generations, while ensuring a balance between economic growth, environmental care and social well-being). That being said, we do see that the liquid absorbing material made of fluffed pulp is beginning to be sourced more sustainably with more sustainable forestry practices. But these emerging sustainability practices, while a step in the right direction, are time consuming. For example, it still requires 5-20 years on average to grow a harvestable Nordic spruce and pine for the pulpwood market.

What is desired, therefore, are sustainable absorbent structures that are comprised of natural materials derived from the flower/fruit/seed/stalk of a plant rather than the pulp from fully matured trees or known SAPs. Such flower/fruit/seed/stalk sustainable absorbent structures have superior absorbency and rewet performance and superior leakage prevention in addition to a soft supple hand feel on both the innermost and outermost layers. In addition, these flower/fruit/seed/stalk structures do not require 15-20 years to harvest, but rather one growing season and perform as well as standard plastic absorbent articles, regardless of biodegradability testing status.

many absorbent articles are starting to incorporate more natural fibers, such as cotton or bamboo, mostly in various blends. While such natural fibers are known to be used in each of the layers, they are typically found in the top sheet. By nature, most of these natural fibers are cellulosic, which tend to absorb and hold liquid rather than quickly transfer the liquid to the core to lock it away from the skin. Therefore, the skin of the wearers of these natural fiber based absorbent articles tends to stay wet and the rewet data shows a much higher likelihood for being rewet than wearers of absorbent articles comprising standard synthetics. Such top sheets need to be improved to more act like a plastic in terms of both rewet and moisture transfer through to the core (otherwise referred to as “strike through”). The objective is to realize a top layer with a fast strike through or strike rate and a low rewet value.

Other absorbent articles, such as feminine hygiene products and incontinence products, have similar constructions as the disposable diaper described above, e.g., they include a top sheet and a core for locking in moisture once it arrives. There are no products currently on the market that meet the needs of those markets with advanced top sheets containing natural fibers that do not retain liquid and keep the skin excessively moist.

U.S. Pat. No. 11,382,801 B2 (the “'801 patent”) discloses top sheet materials with defined apertures with specific angle ranges. However, absorbent articles formed of sheet materials utilizing the apertures disclosed in the '801 patent, with specific angles that form a cone shape, which pushes the moisture from the top sheet though a pore (e.g., using cotton fiber) to the core, does not overcome the shortcomings of using cotton, which absorbs and holds liquid thus swelling to close the pores in the fabric. In the absorbent articles of the '801 patent, liquid can be inhibited from transferring properly to the core, resulting in inadequate performance as an absorbent article, causing leaks and wetness at the wearer-facing openings. For that matter, such absorbent articles and top sheet materials (see, for example, col 8, lines 39-63) are not formed using sustainable materials displaying a fast strike through or strike rate and a low rewet value.

WO 2017/015477 A1 discloses a disposable diaper that is biobased and/or biodegradable. The diaper consisting of a non-woven inner layer comprising natural fibers, a non-woven outer layer comprising natural fibers, and a hydrophobic treatment and a core comprising natural fibers or fibrous material positioned between the inner layer and the outer layer. But while the non-woven inner layer consists of 100% cellulosic fibers, it nevertheless has poor performance as it absorbs and holds up to 150 times its weight before transferring the excess liquid to the core. This keeps the skin of the wearer excessively wet, rather than transferring the majority of the liquid it to the core to lock it away. The disposable and biodegradable diaper also performs poorly in relation to rewetting, where the moisture travels through the inner layer, to the core and when the wearer puts any weight on the structure. For example, when a baby sits down, the liquid travels back up to the inner layer where it is held next to skin, causing poor performance and leakage.

There is no system and/or technology put in place to address performance issues as likely performance enhancing technology will cause a poor hand feel. The concern for the hand feel is not addressed against the wearer but is a concern for the outermost layer of the garment in that teaching. For that matter, due to fact that the disposable and biodegradable diaper disclosed in WO 2017/015477 A1 includes a core of loose fluff cotton and SAP, loose fluff cotton of too short a staple length causes linting. This lint can transfer through the diaper top sheet and not only adhere to the baby's skin but also be released into the air and inhaled causing lung damage. And such loose fluff and SAP alone allow the SAP to settle at the lowest point in the diaper, causing bulky agglomeration of SAP particles in the center of the diaper. This results in both decreasing the maximum absorbency level and discomfort to the baby wearing a diaper that sags down significantly in the center.

WO 2016/023016 A1 discloses a disposable diaper that is biobased and/or biodegradable. The diaper comprises a biobased and/or biodegradable outer sheet impermeable to aqueous medium, a biobased and/or biodegradable inner sheet permeable to aqueous medium, an absorbent pad made of natural fibers and biobased superabsorbents, biobased and/or biodegradable side sheets, fastening tabs and an elastic waist strip. In particular, the outer sheet, inner sheet, side sheets, fastening tabs and fastening mat are said to be made of a thermoplastic polymer selected from the group consisting of aliphatic polyester, aromatic polyester, cellulosic fiber, nonwoven material and combinations thereof. Superabsorbents can be cellulosic based, starch based or protein based. But these known materials are sustainable materials displaying a fast strike through or strike rate and a low rewet value.

Another issue with certain thermoplastic polyesters and cellulosic fibers, such as those disclosed in WO 2016/023016 A1, is that most if not all the thermoplastics are not sustainable. Nor are the biodegradable treatments on the natural fibers sustainable, even though they may be biodegradable and engineered to pass ASTM Test Method D5511-12, ASTM Test Method 5338.92 or ISO CD Test Method 14855 as the reference teaches. For that matter, WO 2016/023016 A1 does not even refer to the issues of natural fibers and their poor rewet properties and high tendency for cellulosic materials to absorb and not transfer the liquid efficiently to the core, still less offer a solution for these issues.

WO 2018020061A1 discloses a hygiene product for personal use that uses a cotton core with a plurality of microcapsules that contain active ingredients that release onto the skin. However, it is not desirable to have SAP or the ingredients contained therein deposited out of the core and onto the skin of the user. Rather, it is more desirable to keep the SAP in place and inside the core, where the moisture is held away from the skin surface.

For that matter, the inventor is unaware of any diaper products that actually contain a sustainable, non-woven hydrophobic inner layer comprising natural materials derived from the flower/fruit/seed/stalk of a plant, which are of course sustainable and that also comprise a plurality of pores, which inner layer also displays a low moisture absorbency within and good rewet performance.

### SUMMARY OF THE INVENTION

Accordingly, the present invention provides sustainable, absorbent structures comprised of natural materials derived from the flower/fruit/seed/stalk of a plant, which overcome the shortcomings of the prior art.

In one embodiment, these inventive, sustainable, absorbent structures have a hydrophobic top sheet that provides superior absorbency due to its character of the pushing liquids to the core rather than allowing the liquids to stay on the surface, as in a conventional cellulosic based top sheet structures. The inventive hydrophobic top sheet also displays good, low rewet performance as well as superior leakage prevention in addition to a soft supple hand feel.

In addition, using flower/fruit/seed/stalk materials, rather than tree-based fiber, obviates a material that while sustainable, could take 15-20 years to harvest. By contrast, flower/fruit/seed/stalk materials are realized in one growing season and known to mimic or exceed the performance of a standard plastic absorbent article, while nevertheless being sustainable, regardless of biodegradability testing status.

The inventive top sheet comprising natural materials derived from the flower/fruit/seed/stalk of a plant that also contains a plurality of pores and a hydrophobic property that does not interfere with the sustainability of the top sheet itself but enhances its rewet characteristics so that it performs more like that of standard plastic unsustainable top sheets while adding comfort for the wearer.

In a preferred embodiment, the top sheet is comprised entirely or substantially of sustainable materials that in diaper form has a rewet of less than 40 grams and a strike through faster than 8 seconds using the INDA single strike through, re-wet, and multiple strike through standardized testing methods. And such absorbent articles, including diapers also have a soft hand feel on its inward facing surface.

The inventive top sheet for use in disposable absorbent articles preferably comprises substantially all natural materials derived from the flower/fruit/seed/stalk of a plant, and preferably is hydrophobic. Such top sheet also preferably comprises a plurality of pores.

In a further preferred embodiment, the inventive top sheet for use on disposable absorbent articles includes a non-woven inner layer comprising natural materials derived from the flower/fruit/seed/stalk of a plant that is either unbleached, to maintain the hydrophobic properties offered by intact lignin, and/or treated to include at least one compound selected from the group consisting of waxes, urethanes, silicones, fluorocarbons, non-fluorochemical repellants applied to at least one surface thereof.

In certain cases, the treatment is applied to the inner layer at 0.0015% to 2.5% solids by weight add on. In certain advantageous embodiments treatment is applied at about 1.55% by weight add on. In some embodiments, the hydrophobic treatment is applied only to the top surface of the inner layer. In certain other embodiments, the treatment is applied to the bottom and top surface of the inner layer.

In certain embodiments, there is no treatment applied to the inner layer rather it is comprised of unbleached cellulosic fibers blended with synthetic fibers such as polyethylene, polypropylene, polyester or other thermoplastic polymer matrix materials. In certain embodiments, the cellulosic content is 10% or greater.

In certain preferred embodiments, the inner layer of the absorbent article is treated with a dendrimer wax. In some of those embodiments, the treatment is applied to a top surface of the inner layer. In some embodiments, the treatment is applied to a top and a bottom surface of the inner layer.

In certain preferred embodiments of the absorbent article, the inner layer includes a plurality of pores.

Absorbent articles so comprised may be a diaper, a nappy, absorbent underpants, training pants, adult incontinence products, pet incontinence products, feminine hygiene products, wound dressings, or breast pads.

The invention also comprises a sustainable diaper formed with a non-woven inner layer comprising cotton treated with a hydrophobic agent selected from the group consisting of waxes, urethanes, silicones, fluorocarbons, and non-fluorochemical repellants and combinations thereof, an outer layer comprising cotton, and an absorbent core comprising cotton and a polyacrylate superabsorbent particles positioned between the inner layer and the outer layer.

In some cases, the diaper has a treatment consisting of dendrimer wax applied to the inner layer. In certain of those cases, the treatment is applied to the inner layer at 0.0015% to 2.5% solids by weight add on. In certain advantageous embodiments the dendrimer wax is applied at about 1.55% by weight add on. In some embodiments, the hydrophobic treatment is applied only to the top surface of the inner layer.

In some cases, the diaper has a treatment consisting of a paraffin wax applied to the inner layer. In certain of those cases, the treatment is applied to the inner layer at 0.5% to 1.2% solids by weight add on. In certain advantageous embodiments the paraffin wax is applied at about 0.75% by weight add on. In some embodiments, the hydrophobic treatment is applied only to the top surface of the inner layer.

In certain embodiments, the diaper has a rewet of less than 40 grams and a strike through faster than 8 seconds using the INDA single strike through, re-wet, and multiple strike through standardized testing methods. Typically, the strike through and re-wet properties have an inverse relationship. When the strike through is fast, the re-wet is higher and vice versa.

Table 1 that follows presents multiple strike through data generated using INDA standardized testing methods and compares the dendrimer treated top sheet in a diaper to commercially available plastic diapers.

Performance is considered acceptable when the Multiple Strike through is less than 6 seconds.

Table 2 that follows presents Single Strike Through data that was generated using INDA standardized testing methods and compares the dendrimer treated top sheet in a diaper to commercially available plastic diapers. Performance is considered acceptable when the Single Strike through is less than 6 seconds.

Table 3 that follows presents re-wet data that was generated using INDA standardized testing methods and compares the dendrimer treated top sheet in a diaper to commercially available plastic diapers. Performance is considered acceptable when the re-wet is less than 0.25 grams.

Table 4 that follows presents multiple Strike Through data that was generated using INDA standardized testing methods and compares the dendrimer treated top sheet fabric alone to untreated 100% cotton fabric.

Table 5 that follows presents single Strike Through data that was generated using INDA standardized testing methods and compares the dendrimer treated top sheet fabric alone to untreated 100% cotton.

Table 6 that follows presents re-wet data was generated using INDA standardized testing methods and compares the dendrimer treated top sheet fabric alone to untreated 100% cotton fabric.

These and other goals and objects of the invention and the particular features and advantages of the invention will become more apparent from consideration of the following drawings and accompanying detailed description.

### BRIEF DESCRIPTION OF THE DRAWINGS

FIG. 1 is a perspective view of a preferred absorbent article in the form of a diaper made in accordance with the present invention;

FIG. 2 is a cross-sectional view of a diaper according to a first embodiment;

FIG. 3 is a cross-sectional view of a diaper according to a second embodiment;

FIG. 4 is a cross-sectional view of a diaper according to a third embodiment; and

FIG. 5 is a perspective view of different embodiment of a diaper made in accordance with the present invention, illustrated in FIG. 1.

### DETAILED DESCRIPTION OF THE INVENTION

The following definitions and methods are provided to better define the present disclosure and to guide those of ordinary skill in the art in the practice of the present disclosure. Unless otherwise noted, terms are to be understood according to conventional usage by those of ordinary skill in the relevant art.

As used in the description, the terms “top,” “bottom,” “above,” “below,” “over,” “under,” “above,” “beneath,” “on top,” “underneath,” “up,” “down,” “upper,” “lower,” “front,” “rear,” “back,” “forward” and “backward” refer to the objects referenced when in the orientation illustrated in the drawings, which orientation is not necessary for achieving the objects of the invention.

Growing season means within a 12-month period.

Harvested seasonally means the flower, fruit, seed, stalk, stem, is removed from the plant or soil within 12 months from emergence.

As used herein the term “nonwoven” fabric or web means a web having a structure of individual fibers or threads which are interlaid, but not in an identifiable manner as in a knitted or woven fabric. Nonwoven fabrics or webs can be formed by various processes including, but not limited to, meltblowing processes, spunbonding processes, spunlacing processes and bonded carded web processes.

As used herein the term “spunbond fibers” refers to small diameter fibers of mechanically and/or eductively drawn polymeric material. Spunbond fibers are generally formed by extruding molten thermoplastic material as filaments from a plurality of fine capillaries of a spinneret with the diameter of the extruded filaments then being rapidly reduced. Examples of spunbond fibers and methods of making the same are described in, by way of example only, U.S. Pat. No. 4,340,563 to Appel et al., U.S. Pat. No. 5,382,400 to Pike et al., and U.S. Pat. No. 5,795,926 to Pike et al.; the entire content of the aforesaid patents is hereby incorporated by reference herein. Spunbond fibers are generally not tacky when they are deposited onto a collecting surface and are continuous.

As used herein “spunlaced fibers” refers to those made by a process involving the cohesion and the interlacing of the elementary fibers with one another, typically by means of a plurality of jets of water under pressure passing through a moving fleece or cloth and causing the fibers to intermingle with one another. As an example, a process for the production of spunlaced nonwoven cloths has been described in U.S. Pat. Nos. 3,214,819, 3,485,706 and 3,508,308; the entire content of the aforesaid patents is incorporated by reference herein.

As used herein the term “meltblown fibers” means fibers of polymeric material which are generally formed by extruding a molten thermoplastic material through a plurality of die capillaries as molten threads or filaments into converging high velocity air streams which attenuate the filaments of molten thermoplastic material to reduce their diameter. Thereafter, the meltblown fibers can be carried by the high velocity gas stream and are deposited on a collecting surface to form a web of randomly dispersed meltblown fibers. Meltblowing processes are disclosed in, by way of example only, in U.S. Pat. No. 5,271,883 to Timmons et al.; U.S. Pat. No. 5,160,746 to Dodge et al.; U.S. Pat. No. 4,526,733 to Lau; U.S. Pat. No. 5,652,048 to Haynes et al.; and U.S. Pat. No. 5,366,793 to Fitts et al.; the entire contents of the aforesaid patents are hereby incorporated by reference herein. Meltblown fibers are generally smaller than about 10 micrometers in average diameter and, unlike spunbond fibers, are generally tacky when deposited onto a collecting surface, thereby bonding to one another during the deposition step.

The word “disposable” is used in the specification to describe absorbent articles that are not intended to be laundered or otherwise restored or reused as an absorbent article. They are intended to be discarded after a single use and preferably to be disposed of in an environmentally friendly manner.

The phrases “personal product,” “absorbent product” and “absorbent article” are used interchangeably herein and are intended to include products or articles used to absorb waste body fluids or other body waste, and includes diapers, nappies, absorbent underpants, training pants, adult incontinence products, pet incontinence products, feminine hygiene products, wound dressings, and breast pads. It will be appreciated that the above list is not limiting and the teachings of the present invention can be implemented into other types of products that are intended for fluid absorption.

The phrase “foam structure” is intended to include products or articles made by a process of mixing oil-based chemicals together, which react and change from a liquid to a solid state. “Foam” can be injected into a mold or formed in big block structures. Foams can be flexible and porous substances made from a natural or synthetic compounds with various ingredients and whipped into a froth that then result in a product that is 80-85% air and 15% solids after formation.

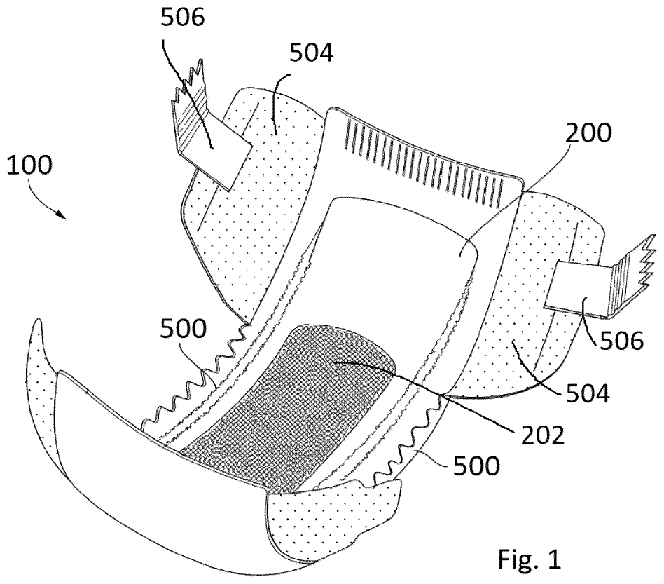

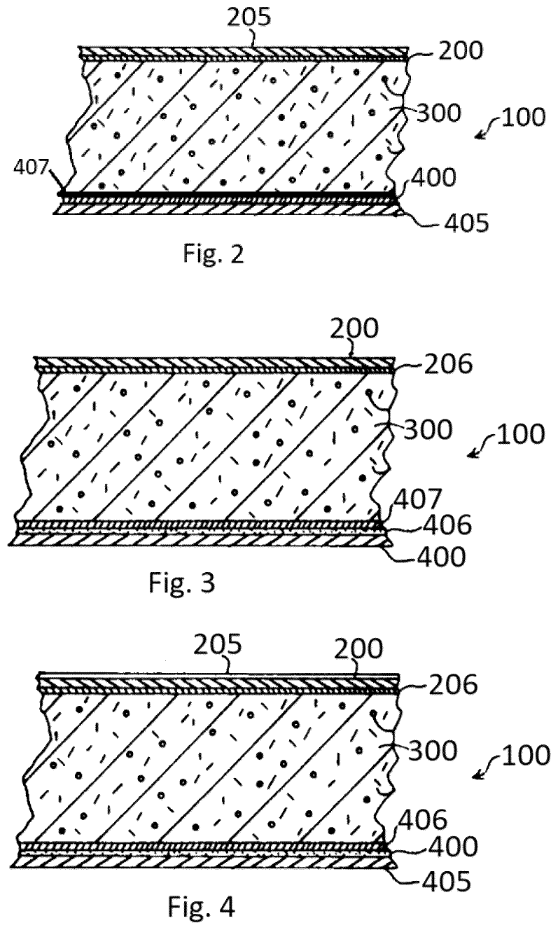

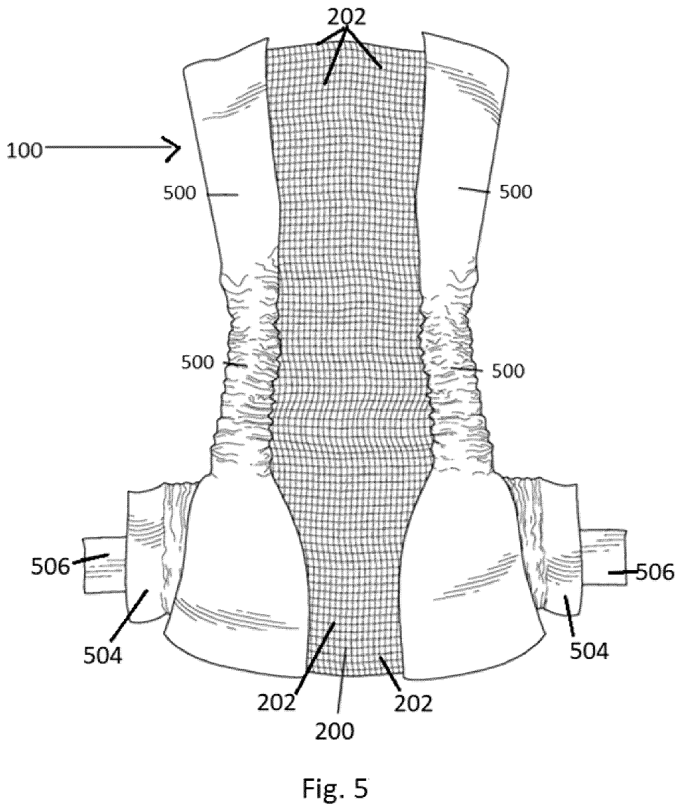

FIGS. 1 and 2 illustrate an absorbent article (personal product) 100 according to one exemplary embodiment and, in particular, the absorbent article 100 is in the form of a disposable diaper for an infant (baby). It will be understood that the illustrated diaper 100 is only an exemplary embodiment and the teachings of the present invention can be implemented in a toddler training pant/diaper, other similar absorbent products, as well as other absorbent products that may or may not be mentioned herein. FIGS. 1 and 5 are perspective views of different embodiments of a diaper 100, while FIG. 2 is a cross-sectional view of the diaper 100.

The diaper 100 is formed generally of an inner layer (top sheet) 200, an inner acquisition/distribution layer and/or absorbent core 300, a film layer 407 and an outer shell or cover (back sheet) 400, as shown.

In some instances, there are also side panels, otherwise known as leg cuffs 500 (see FIG. 1), which are designed to effectively prevent leakage at the sides of the diaper 100, thereby keeping the skin dry and protected. In some embodiment, the leg cuffs 500 can be constructed as a set of inner standing leg cuffs or gathers 501, as well as the (outer) leg cuffs 500. As shown in FIG. 1, the leg cuffs 500 or gathers 501 (not shown) are generally formed in the central portion of the diaper 100.

In one exemplary embodiment, each leg cuff 500 is formed of a nonwoven material that contains fibers derived from the flower/fruit/seed/stalk of a plant. In the FIG. 1 diaper, each leg cuff 500 is preferably formed of a nonwoven hydrophobically treated cotton structure with a spandex inlay to provide the desired elasticity. When the diaper 100 includes a defined elastic waistband, it too can be formed of a nonwoven material that contains fibers derived from the flower/fruit/seed/stalk of a plant, such as cotton structure with a spandex inlay to provide elasticity thereto.

It will be appreciated that the elastic structure can be formed of other materials, such as spandex and other synthetics. As mentioned herein, the elastic structures are used in leg cuffs and also, they can be used in lateral side panels and in tape constructions. For that matter, many gasketing cuffs use spandex to provide a seal with the infant's legs.

In preferred embodiments, the leg cuffs 500 are comprised of cotton and spandex with a hydrophobic treatment. In order to keep the diaper 100 attached, and substantially sealed against the wearer's body, flaps 504 with fasteners 506 are included. The fastener's include a biocompatible adhesive coating, such as pressure sensitive adhesives, dissolvable adhesive films, such as those derived from acrylate, silicone, polyurethane, natural rubber, hydrocolloids and hydrogels. Rubber adhesives are sturdy and tolerate moisture, are weather resistive.

### Inner Layer

As previously mentioned, the inner layer 200 plays an important role in the construction of the diaper since the inner layer 200 is the layer that is next to the most sensitive areas of the baby's body, sometimes for hours at a time. The quality and makeup of the inner layer 200 layer also determines the level of absorbency, rewet, strikethrough and comfort (softness) for the baby.

In accordance with the present invention, the inner layer 200 is formed from natural materials derived from the flower/fruit/seed/stalk of a plant (e.g., 100% organic cotton or coconut). More specifically, the inner layer 200 comprises a non-woven fabric (material) that is formed from natural fibers derived from the flower/fruit/seed/stalk of a plant.

Any number of different natural fibers derived from the flower/fruit/seed/stalk of a plant can be used to form the non-woven inner layer 200 so long as they are suitable for the intended use described herein. Exemplary natural fibers derived from the flower/fruit/seed/stalk, include but are not limited to limited to: cotton, linen, coconut, flax, bamboo, etc.

Cotton is the most widely used natural fiber and is very absorbent, easy to care for, and comfortable for babies. Cotton is almost pure cellulose and is both soft to the touch and advantageously has breathability. As with many materials, cotton is available and marketed in an organic form which is generally understood to mean that the cotton is grown without the use of any synthetic agricultural chemicals, such as fertilizers or pesticides.

Bamboo is a sustainable crop that can readily supply the natural fibers. Bamboo's quick growth cycle allows it to be replaced quickly with a new crop upon harvesting. Bamboo does not typically require fertilizers or pesticides to grow. Bamboo is nice for diapering because it is soft and very absorbent where processed therefore.

Linen is a sustainable crop. Like bamboo, linen's quick growth cycle allows it to be replaced quickly with a new crop upon harvesting. As for wicking, linen can hold 20% of its weight in water before feeling wet, and requires approximately 1/5 of the pesticides and fertilizers that cotton requires to grow. Linen produces long fibers which offer little to no lint and are non-static, insect repellent, non-allergenic and fairly stain-resistant. Linen fibers also have antibacterial properties.

It will also be appreciated that the inner layer 200 can be formed as a blend of two or more of the above-mentioned natural materials.

It will also be appreciated that the inner layer 200 can be formed as a blend of any of the above-mentioned natural materials and thermoplastic polymers such as polyethylene, polypropylene, polyester, et al.

Most cotton-based personal products/absorbent articles preferably are constructed of layers formed using spunbond or spunlaced fibers.

The inner layer 200 can be either hydrophobically treated on one side and hydrophilically treated on the other or hydrophobically treated on just one or both sides. More particularly, the non-woven material forming the inner layer 200 can undergo a surface treatment using a treatment composition which either imparts hydrophilic properties or hydrophobic properties. Any number of different surface treatments can be used to impart increased hydrophilicity to the nonwoven fabric. For example, chemicals, such as surfactants, wetting agents and rewetting agents can increase the hydrophilic nature of the material and can make a fabric that is naturally hydrophobic into one that is hydrophilic. With respect to fibers, natural fibers tend to be hydrophilic, especially when natural oils have been removed from them. The surface treatment can be performed using any number of suitable techniques and more specifically, conventional treatments involve steps such as dipping the nonwoven in a treatment bath, coating or spraying the nonwoven with the treatment composition, and printing the nonwoven with the treatment composition.

When a nonwoven web is formed of a hydrophobic material or otherwise exhibits hydrophobic properties, it is often desirable to modify the surface of the nonwoven web using a hydrophilic surfactant to increase the wettability of the web. To meet this end, an external hydrophilic surfactant is typically applied to the surface of the nonwoven web. An internal hydrophilic surfactant is typically blended with the material (e.g., cellulose fibers) used to form the nonwoven web, and later migrates to the surface after the nonwoven web is formed. External and internal hydrophilic surfactants may be characterized in terms of their durability and wettability.

The durability of a surfactant refers generally to its ability to withstand stresses, such as repeated washing cycles of the nonwoven fabric, without being removed from the fabric or otherwise losing its effectiveness. The wettability of a surfactant refers generally to its ability to transform a hydrophobic nonwoven web into a fabric which readily assimilates and distributes aqueous liquids. Surfactants which cause an otherwise hydrophobic nonwoven web to assimilate liquids at a relatively fast pace, with high fluid intake volumes, are referred to as faster wetting surfactants. Surfactants which cause the nonwoven web to assimilate aqueous liquids at a relatively slow pace, with low fluid intake volume, are referred to as slower wetting surfactants.

The surface treatment of the top sheet layer is configured to increase the hydrophobic nature of the nonwoven by increasing the surface tension of the nonwoven, increasing the contact angle with the liquid and allowing the fluid to be fully repellent when presented on the top sheet, if the fluid is introduced to the top sheet independently of the full diaper structure.

As shown in FIG. 1, and which can be applied in other embodiments, the inner layer 200 comprises a plurality of openings or pores 202, such as those provided in a gauze-like material. These openings (pores 202) readily allow for the passage of fluid into an inner acquisition/distribution layer and/or absorbent core 300 (FIG. 2) so that fluid is not retained on the surface of the nonwoven inner layer 200 that touches the skin.

The nonwoven inner layer 200 can be formed by any number of suitable processes including, but not limited to, hydroentanglement, carding, air-laying, thermal treatment, or other suitable process. Pores, a dot matrix, or perforations can be created during the nonwoven formation process or can be applied to the nonwoven after formation. Any number of means can be employed to create these openings (e.g., pores 200) in a pre-formed nonwoven, including but not limited to air jet formation, laser formation, during extrusion or casting, or by passing a sheet through a roller mill wherein the rollers have microspikes suitable for forming pores on the sheet. Additionally, a porous sheet can be formed by incorporating microparticles of a water-soluble substance and passing the sheet through a water bath after its formation. The water dissolves the microparticles so as to produce pores. In particularly preferred embodiments, the pores are structurally built during cotton sheet formation.

The openings or pores 220 in the nonwoven inner layer 200 may be any shape that allows the passage of fluid, preferably oval or round. The openings are typically between about 0.07 cm and about 0.40 cm in diameter. Preferably, the pores 202 of the nonwoven inner layer 200 are 0.18 cm to 0.23 cm in diameter.

In addition, both the plurality of pores 202 and the hydrophobic nonwoven top sheet 200 create a system of which increases the rewet performance and maintains the fluid near the core 300 of the diaper preventing wetness from reaching back up through the diaper to the skin. The surface treatment of the top sheet 200 is thus configured to increase the hydrophobic nature of the nonwoven by increasing the surface tension of the nonwoven, increasing the contact angle with the liquid and allowing the fluid to repel. This tension is at such a level that stand alone the top sheet with the plurality of pores will hold a bead of water and will not allow the water to go through the pores due to the high surface tension and contact angle on its on merit without a superior fast wetting surfactant located on the inner surface of the top sheet itself or below the top sheet structure in either in the ADL layer, core wrap or core itself.

FIG. 2 shows a surface treatment 205 applied to a top surface of the nonwoven inner layer 200, i.e., the surface in contact with skin. FIGS. 3 and 4 present alternative embodiments that both show surface treatment 206 applied to a bottom surface of the nonwoven inner layers 200, i.e. the surface closer to the respective cores 300. The FIG. 4 embodiment includes that the surface treatment 205, 206 is applied to both the top and bottom surfaces of the inner layer 200.

It will also be understood that the surface treatment applied to the inner layer 200 can be configured as a finish that imparts hydrophobic properties on either one side and the other side is left in its natural state, as a finish that imparts hydrophobic properties on one side and as a finish that imparts hydrophilic properties the other side, or hydrophobic on both sides of the inner layer.

In some embodiments, the treatment may comprise Finish RPW dendrimer wax dispersion, Nylwick (or Nylwck) modified water dispersible polyester with ethoxylated alcohols, Aquatek Uno sulfonated nylon, Block S sulfonated nylon, WSR XF non-fluorine water repellant, NF-21 fluorinated (C6) surfactant, HCO16 and/or ethox 2191. A particularly preferred form is provided as Finish Nylwck by Phoenix Chemical Company. In certain preferred embodiments, the treatment may comprise Permaseal WSR-XF C6 fluorocarbon and/or Flexiwet NF non-fluorocarbon silicon water repellant.

In certain embodiments, the hydrophobic treatment comprises dendrimer wax. In certain of those embodiments, the treatment comprises RPW. Dendrimer waxes that may be used in the invention are available in liquid form and can be applied to nonwoven materials by a number of techniques readily known in the art. An advantage of wax-based treatments is the mitigated environmental impact of wax as opposed to fluorocarbons.

In one embodiment, the treatment is applied to the natural fibers at 0.0015% to 2.5% solids. In some embodiments, 0.005% to 0.75% solids of the treatment is applied. In certain of those embodiments, about 0.25% by weight of the treatment is applied. In particularly preferred embodiments, 0.005% to 0.35% by weight of the treatment is applied to fabric. It is important that at least 0.0015% add on by weight of the hydrophobic treatment is applied to the outer layer. It is believed that the use of lower levels of the stated ranges of such ingredients allow the treated fabrics to retain hydrophilic characteristics, as opposed to the water resistant and water repellant properties imparted by higher concentrations of such chemicals to fabrics.

In some particularly preferred embodiments, the invention comprises applying about 1.55% by weight of finish RPW dendrimer wax to a nonwoven cotton or cotton elastomeric blend. It is particularly advantageous to apply a treatment comprising dendrimer wax or a dendrimer wax emulsion to the inside (top) surface of the outer sheet.

As used herein, the terms “percent”, “%,” “solids percent” and “wt %” all mean the percentage by weight of the indicated component or ingredient within the product or composition in which it is present, without dilution after drying, unless otherwise indicated by the context in which the term is used. When the treatment is applied to a fabric, the percent”, “%,” “solids percent” and “wt %” refers to the amount applied to the fabric upon drying unless otherwise stated.

Treatment solutions and suspensions comprise various percentages by weight of the treatment compositions in water unless another solvent/diluent is indicated.

An external hydrophobic agent is applied to the surface of the nonwoven web. An internal hydrophobic agent is blended with the material used to form the nonwoven web, and later migrates to the surface after the nonwoven web is formed. External and internal hydrophobic agents can be characterized in terms of their durability and wettability similar to hydrophilic agents as described above.

FIG. 2 also highlights a surface treatment 205 applied to the top surface of the nonwoven top sheet 200, i.e., the surface next to the skin. The FIG. 3 embodiment highlights surface treatment 206 applied to the bottom surface of the top sheet 200, i.e., the surface closer to the core 300, where the FIG. 4 embodiment highlights the surface treatment 205, 206 applied to both the top and bottom surfaces of the top sheet 200.

As mentioned above, a surface treatment can be applied to the inner layer 200 to impart hydrophobicity.

The inner layer 200 is thus a soft, comfortable hydrophobic layer formed of a sustainable material derived from the flower/fruit/seed/stalk of a plant that resists moisture regain and does not hold liquid next to the surface of the absorbent article but rather transfers it to the core where it is held.

### Leg Cuffs

As introduced above, the diaper embodiments may also include a pair of leg cuffs 500 that are designed to effectively prevent leakage at the sides of the diaper 100. The leg cuffs keep the infant's skin dry and protected. Currently, the leg cuffs are hydrophobic plastic materials made form polyethylene, polypropylene, or polyester. As with the other components, the leg cuffs 500 can be formed of a sustainable material derived from the flower/fruit/seed/stalk of a plant. Any number of sustainable materials, including those described herein, can be used to form the leg cuffs 500. For example, the leg cuffs 500 can be formed of cotton (nonwoven) or other coconut material.

Each leg cuff 500 has an associated elastic element to ensure improved fit around the legs and prevent leakage. The elastic element may or may not be formed of a sustainable material derived from the flower/fruit/seed/stalk of a plant. As in traditional diapers, the elastic element extends along a length of the leg cuff 500 and creates a cuff gather 501 in a central location of the cuff 500, as illustrated in FIG. 1.

It will also be understood that the surface treatment applied to the leg cuffs layer 500 can be configured as a finish that imparts hydrophobic properties on both sides of the leg cuffs 500. Otherwise, the leg cuffs will hold moisture and will not allow the transfer of liquid to the core to lock it away from the skin.

In some embodiments, the treatment may comprise finish RPW dendrimer wax dispersion, Nylwick (or Nylwck) modified water dispersible polyester with ethoxylated alcohols, Aquatek Uno sulfonated nylon, Block S sulfonated nylon, WSR XF non-fluorine water repellant, NF-21 fluorinated (C6) surfactant, HCO16 and/or ethox 2191. A particularly preferred form is provided as Finish Nylwck by Phoenix Chemical Company. In certain preferred embodiments, the treatment may comprise Permaseal WSR-XF C6 fluorocarbon and/or Flexiwet NF non-fluorocarbon silicon water repellant.

In certain embodiments, the hydrophobic treatment comprises dendrimer wax. In certain of those embodiments, the treatment comprises RPW. Dendrimer waxes typically used in the invention are available in liquid form and can be applied to nonwoven materials by a number of techniques readily known in the art. One advantage of wax-based treatments is that using wax rather than fluorocarbons mitigates environmental impact when compared to fluorocarbons.

The hydrophobic treatment is applied to the natural fibers at 0.018% to 4.75% solids. In some embodiments 0.03% to 2.85% solids of the treatment is applied. In certain of those embodiments, about 0.5% by weight of the treatment is applied. In particularly preferred embodiments, 0.02% to 3.5% by weight of the treatment is applied to fabric. It is important that at least 0.018% add on by weight of the hydrophobic treatment is applied to the leg cuff material. It is understood that use of lower levels of such ingredients allows the treated fabrics to retain hydrophilic characteristics, as opposed to the water resistant and water repellant properties imparted by higher concentrations of such chemicals to fabrics.

In some particularly preferred embodiments, the invention comprises applying about 2.95% by weight of Finish RPW dendrimer wax to a nonwoven cotton or cotton elastomeric blend. In particular, it is advantageous to apply a treatment comprising dendrimer wax or a dendrimer wax emulsion to the inside (top) surface of the outer sheet.

Treatment solutions and suspensions comprise various percentages by weight of the treatment compositions in water unless another solvent/diluent is indicated.

An external hydrophobic agent is typically applied to the surface of the nonwoven web. An internal hydrophobic agent may be blended with the material used to form the nonwoven web, and later migrates to the surface after the nonwoven web is formed. External and internal hydrophobic agents can be characterized in terms of their durability and wettability similar to hydrophobic agents as described above.

In certain preferred embodiments, hydrophobic surface treatments, comprise a wax, a non-fluorocarbon repellant, or a combination thereof. An example of a wax that may be used to treat the leg cuff layer is Finish RPW available from Phoenix Chemical. An example of a non-fluorocarbon repellant that may be used to hydrophobically treat the leg cuff layer is WSRXF available from Phoenix Chemical.

It should be understood that the foregoing is illustrative and not limiting, and that obvious modifications may be made by those skilled in the art without departing from the spirit of the invention. Accordingly, reference should be made primarily to the accompanying claims, rather than the foregoing specification, to determine the scope of the invention.

What is claimed is:

1. A disposable absorbent article having an inner layer, the inner layer comprising:
natural fibers derived from the flower/fruit/seed/stalk of a plant that is harvested in a single growing season; and
a treatment applied to at least one surface thereof at a solid loading percent of 2.5% or less, the treatment comprising at least one compound selected from the group consisting of waxes, urethanes, silicones, fluorocarbons, non-fluorochemical repellants;
wherein the at least one surface comprises a plurality of pores.

2. The absorbent article of claim 1, wherein the absorbent article is selected from the group consisting of: diapers, nappies, absorbent underpants, training pants, adult incontinence products, pet incontinence products, feminine hygiene products, wound dressings, and breast pads.

3. The absorbent article of claim 1, wherein the inner layer treatment comprises dendrimer wax or a dendrimer wax emulsion that has been applied to the inner layer.

4. The absorbent article of claim 1, wherein the inner layer treatment comprises a paraffin wax or a paraffin wax emulsion that has been applied to the inner layer.

5. The absorbent article of claim 1, wherein the inner layer treatment is applied to only a top surface of the inner layer.

6. The absorbent article of claim 1, wherein the inner layer treatment is applied to a top surface and a bottom surface of the inner layer.

7. The absorbent article of claim 1, wherein the inner layer treatment is applied to a top surface of the inner layer and a hydrophilic treatment is applied to a bottom surface of the inner layer.

8. The absorbent article of claim 1, wherein the treatment is applied to the inner layer at 0.0015% to 1.55% by weight add on.

9. The absorbent article of claim 1, further including a hydrophobic leg cuff.

10. The absorbent article of claim 1, further including:
a hydrophobic leg cuff comprising natural fibers derived from the flower/fruit/seed/stalk of a plant that is harvested in a single growing season; and
a repellent treatment applied to at least one surface thereof at a solid loading percent of 4.75% or less and comprising at least one compound selected from the group consisting of waxes, urethanes, silicones, fluorocarbons, non-fluorochemical repellants.

11. The absorbent article of claim 10, wherein the treatment is applied to the leg cuff at 0.018% to 3.5% by weight add on.

12. The absorbent article of claim 10, wherein, the leg cuff treatment comprises dendrimer wax or a dendrimer wax emulsion that has been applied to any surface thereof.

13. The absorbent article of claim 10, wherein, the leg cuff treatment comprises a paraffin wax or a paraffin wax emulsion that has been applied to any surface thereof.

14. A disposable diaper having an inner layer comprising natural fibers and a treatment, the treatment applied to at least one surface of the inner layer and comprising at least one compound selected from the group consisting of waxes, urethanes, silicones, fluorocarbons, non-fluorochemical repellants and applied at a solid loading percent of 2.5% or less, wherein the treatment further comprises a plurality of pores.

15. The diaper of claim 14, wherein the treatment comprises a dendrimer wax or a dendrimer wax emulsion that has been applied to the inner layer.

16. The diaper of claim 14, wherein the treatment comprises a paraffin wax or a paraffin wax emulsion that has been applied to the inner layer.

17. The diaper of claim 14, wherein the treatment is applied to the inner layer at 0.0015% to 1.55% by weight add on.

18. The diaper of claim 14, wherein the treatment is applied to only a top surface of the inner layer.

19. The diaper of claim 14, wherein the treatment is applied to a top surface and a bottom surface of the inner layer.

20. The diaper of claim 14, wherein the treatment is applied to a top surface of the inner layer and a hydrophilic treatment is applied to a bottom surface of the inner layer.

21. The diaper of claim 14, further comprising a leg cuff, the leg cuff comprising:
natural fibers derived from the flower/fruit/seed/stalk of a plant that is harvested in a single growing season;
a treatment applied to at least one surface thereof, the treatment comprising at least one compound selected from the group consisting of waxes, urethanes, silicones, fluorocarbons, non-fluorochemical repellants and applied at a solid loading percent of 4.75% or less.

22. The absorbent article of claim 21, wherein the treatment is applied to the leg cuff at 0.018% to 3.5% by weight add on.

23. The absorbent article of claim 21, wherein, the leg cuff treatment comprises dendrimer wax or a dendrimer wax emulsion that has been applied to any surface thereof.

24. The absorbent article of claim 21, wherein, the leg cuff treatment comprises a paraffin wax or a paraffin wax emulsion that has been applied to any surface thereof.
